(19) United States
(12) Patent Application Publication
(10) Pub. No.: US 2006/0219753 A1
(43) Pub. Date: Oct. 5, 2006

(54) Circumcision apparatus and method using the same

(76) Inventors: Kwok-Wai Chiu, Plano, TX (US); Timothy Chiu, Plano, TX (US)

Correspondence Address: BIRCH STEWART KOLASCH & BIRCH, PO BOX 747, FALLS CHURCH, VA, 22040-0747, US

(21) Appl. No.: 11/095,489

(22) Filed: Apr. 1, 2005

(51) Int. Cl. A61B 17/04

(52) U.S. Cl. 227/180.1

(57) ABSTRACT

A stapler apparatus and a method to perform a surgical operation (circumcision preferred) using the same are provided. The stapler apparatus includes: a staple nail with two legs, a hammer for striking the staple nail, a staple plate for bending the two legs, a knife for cutting a portion of the patient, a member and a pry device. The member has a first end and a second end, with the member being connected with the knife at the first end. The pry device is disposed between the first end and the second end. When a force is applied to the second end of the member, the knife moves to separate the portion into a first part and a second part, and simultaneously, the force is also applied to the hammer to strike the staple nail to approximate a cut on the second part to reduce bleeding.

### FIELD OF THE INVENTION

This invention generally relates to the field of surgical apparatuses and a method using the same. In particular, the invention relates to a circumcision instrument for use in a circumcision surgery.

### BACKGROUND OF THE INVENTION

Circumcision is a well-established surgical procedure that has been practiced for centuries for medical needs or religious purposes. The procedure involves the use of scissors and clamps to cut the penile foreskin and the subsequent use of suture materials to approximate the cut ends of the foreskin. It usually takes approximately 20 to 30 minutes (excluding the time for administration of anesthesia) to finish the procedure. It always causes bleeding during the operation, as well as tissue swelling and edema during and after operation. As a result, current methods of circumcision require a much longer recovery time, and there are more chances of developing potential complications, and the raised possibility of infection. In addition, the cut ends are usually uneven.

### SUMMARY OF THE INVENTION

The object of the present invention is to provide a surgical instrument for performing circumcision on a patient in a newer, faster, easier and safer way.

Another object of the present invention is to provide a surgical instrument for cutting the foreskin and approximating the cut ends, as well as to simultaneously stop any bleeding.

Another object of the present invention is to provide a surgical instrument that uses a hand grasp force to operate.

A further object of the present invention is to provide a surgical instrument that uses a metallic staple instead of traditional suture materials.

Yet another object of the present invention is to provide a surgical instrument that can shorten the operative time.

Still yet another object of the present invention is to provide a surgical instrument that causes much less tissue edema and swelling during and after the operation.

Other objects and advantages of the present invention will become apparent from the following descriptions, taken in connection with the accompanying drawings, wherein, by way of illustration and example, several embodiments of the present invention are disclosed.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1a is a perspective view illustrating an embodiment of the stapler apparatus of the present invention;

FIG. 1b is a perspective view illustrating another embodiment of the stapler apparatus of the present invention;

FIGS. 2a, 2b and 2c are schematic views illustrating the method of operation of the embodiment as shown FIG. 1a;

FIG. 3a shows one embodiment of the stapler apparatus of the present invention;

FIG. 3b shows another embodiment of the stapler apparatus of the present invention; and

FIGS. 4a, 4b, 4c and 4d are schematic views illustrating the circumcision method using the stapler apparatus of the present invention.

### DETAILED DESCRIPTION OF THE INVENTION

A stapler apparatus 100 and a method to perform a surgical operation using the same are provided. The stapler apparatus 100 can also be applied to surgical procedures such as hemorrhoidectomy or skin lesion excision. Furthermore, the stapler apparatus 100 is preferred, but not limited, to perform a circumcision.

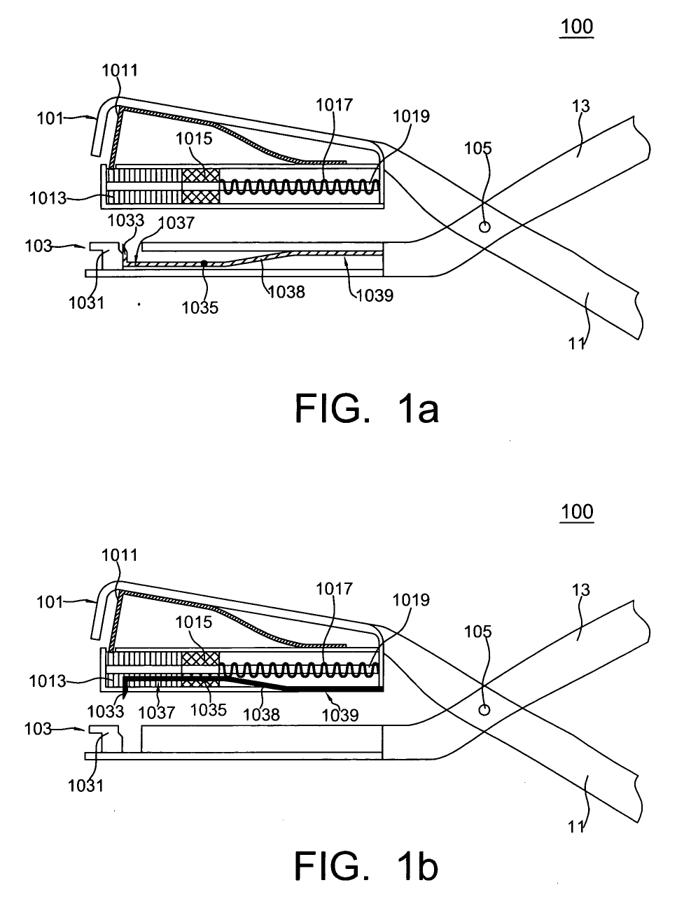

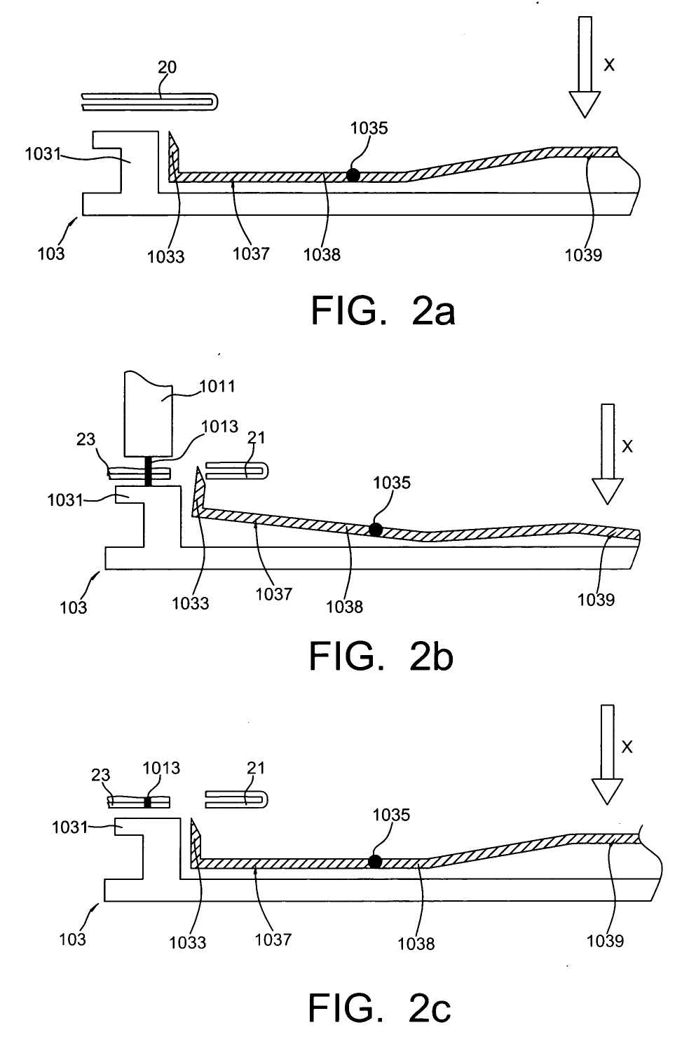

Referring to FIG. 1a, the stapler apparatus 100 includes a first apparatus 11 and a second apparatus 13. The first apparatus 11 is pivotally connected with the second apparatus 13 by a connection device 105. The first apparatus 11 includes a hand stapler assembly 101, and the second apparatus 13 includes a cutter assembly 103. The hand stapler assembly 101 includes at least one staple nail 1013, which has two legs each, a hammer 1011 for striking the staple nail, a block 1015 used to push the staple nails 1013 forward, a spring 1017 and a shaft 1019 both used to maintain an appropriate position of the block 1015. The cutter assembly 103 includes a staple plate 1031 for bending the two legs, a knife 1033 for cutting the foreskin of a patient, a member 1038, and a pry device 1035. The member 1038 has a first end 1037 and a second end 1039, and the member 1038 is connected with the knife 1033 at the first end 1037. A user can operate the stapler apparatus 100 by way of hand grasping, like the operational usage of a stapler or a pair of scissors. Details are referring to the following descriptions.

Now please refer to FIG. 1b, which shows another embodiment of the stapler of the present invention. The stapler apparatus 100 includes a first apparatus 11 and a second apparatus 13, and the first apparatus is pivotally connected with the second apparatus 13 by a connection device 105. The first apparatus 11 includes a hand stapler assembly 101, and the second apparatus 13 includes a support assembly 103. The hand stapler assembly 101 includes at least one staple nail 1013, which has two legs each, a hammer 1011 for striking the staple nail, a block 1015 used to push the staple nails forward, a spring 1017 and a shaft 1019 both used to maintain an appropriate position of the block 1015. The hand stapler assembly 101 also includes a knife 1033 for cutting the foreskin of a patient, a member 1038, and a pry device 1035. The member 1038 has a first end 1037 and a second end 1039, and the member 1038 is connected with the knife 1033 at the first end 1037. The support assembly 103 only includes a staple plate 1031 for bending the two legs. The difference between this embodiment and the aforementioned embodiment in FIG. 1a is that the knife 1033, the pry device 1035 and the member 1038 are disposed in the hand stapler assembly 101. A user can utilize the stapler apparatus 100 by hand grasping, like the operational usage of a stapler or a pair of scissors. Details are referring to the following descriptions.

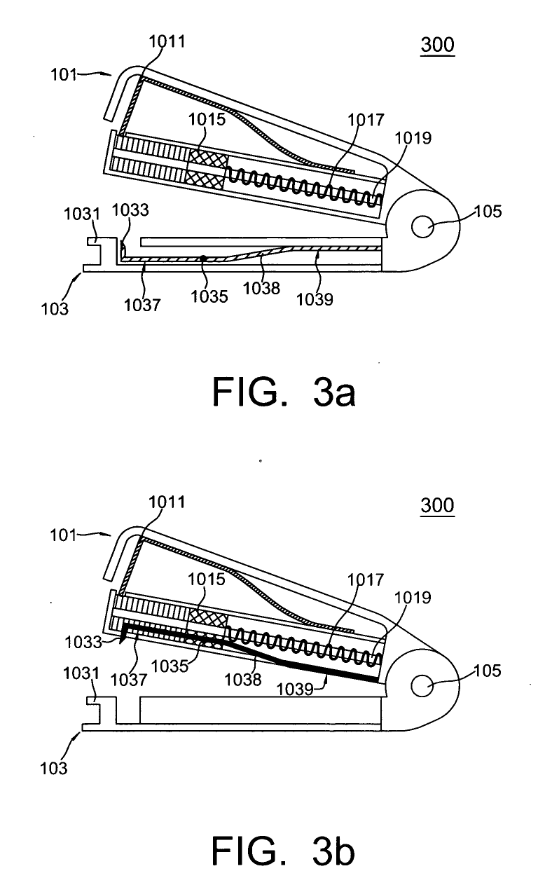

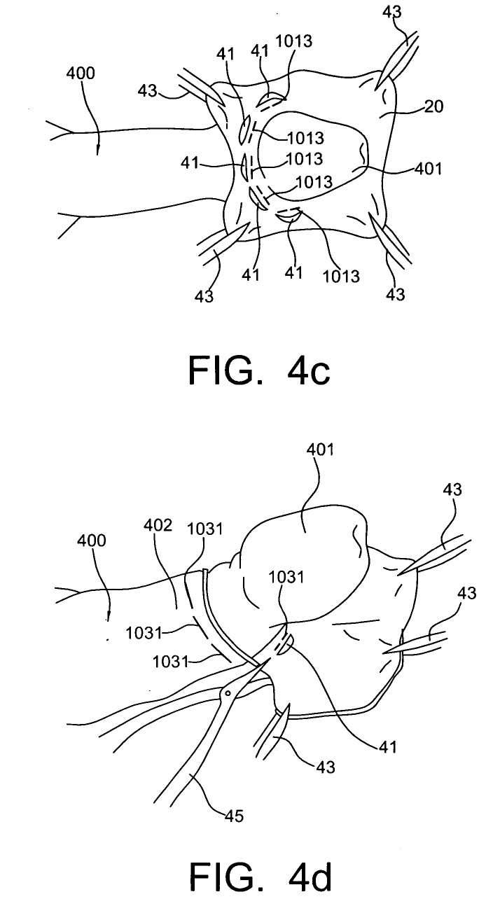

FIGS. 2a, 2b and 2c are schematic views illustrating the method of operation of the embodiment of FIG. 1a. Referring now to FIG. 2a, the foreskin 20 of a patient is placed at an operational position (more details referring to FIGS. 4a-4c are described below). The foreskin 20 to be circumcised should be placed on the area above the staple plate 1031 and the knife 1033. The foreskin 20 has two layers as shown in FIG. 2a. More specifically, the predetermined location to be cut on the foreskin 20 should be placed right above the knife 1033. When the foreskin 20 to be circumcised is in place, a user can prepare to perform the circumcision. Referring now to FIG. 2b, a user grasping the stapler apparatus 100 can apply a downward force (X direction in the FIG. 2b) on the second end 1039 of the member 1038, causing the knife 1033, which is disposed at the first end 1037 of the member 1038, to rise to separate the foreskin 20 into a first part 21 and a second part 23. Simultaneously, the downward force is also applied to the hammer 1011 to strike the staple nail 1013 to make the two legs penetrate the second part 23. Please refer to FIG. 2c. As the downward force keeps being applied to the hammer 1011, the staple plate 1031 starts to bend the two legs of the staple nail 1013 in order to approximate the cut ends on the second part 23 and also to stop bleeding. Thus, the staple nail 1013 approximates the two layers of the foreskin 20 together. When the downward force no longer exists, the hammer 1011 returns to its original position, and a cut 41 (referring to FIG. 4b) is formed between the first part 21 and the second part 23 of the foreskin 20. Detailed descriptions of the method utilizing the staple apparatus 100 to perform circumcision will be elaborated in FIGS. 4a-4d. The detailed operation method of the embodiment described in FIG. 1b is similar to the operation method of the embodiment described in FIG. 1a.

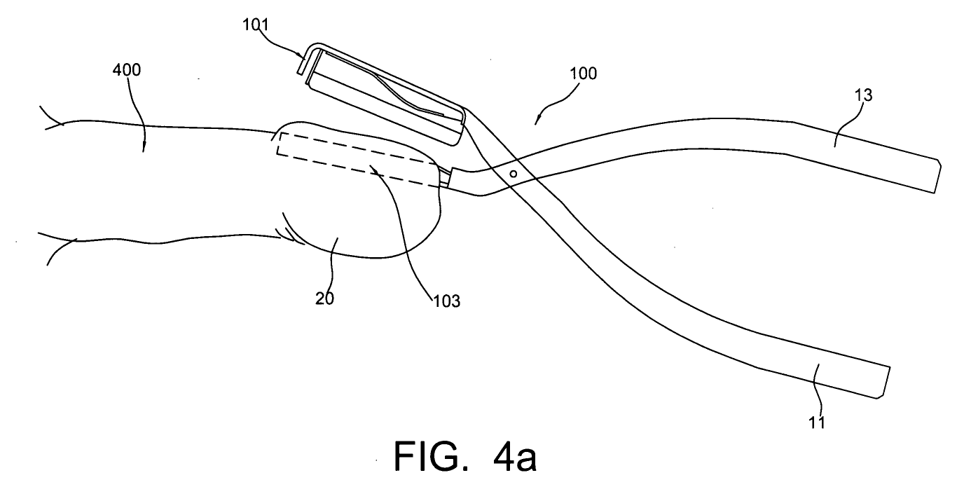

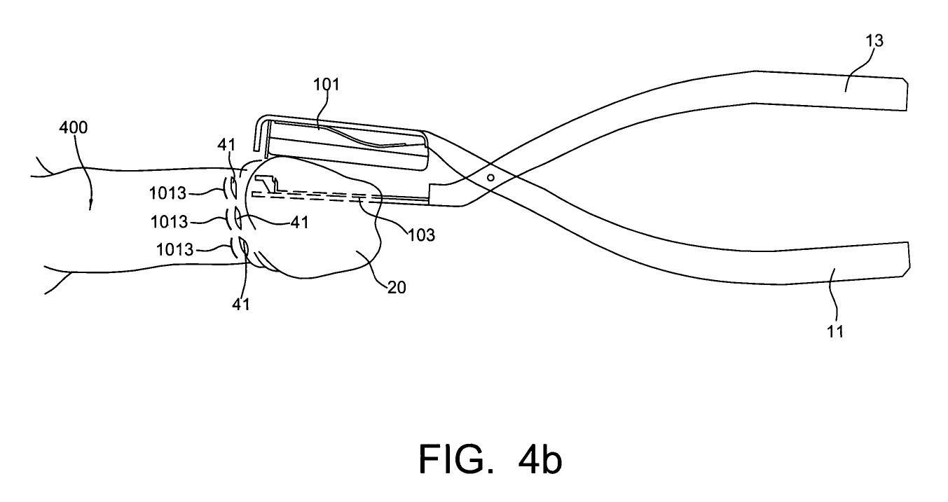

FIG. 3a shows another embodiment of the stapler apparatus of the present invention. In this embodiment, the stapler apparatus 300 includes a hand stapler assembly 101 and a support assembly 103. A connection device 105 pivotally connects the hand stapler assembly 101 and the cutter assembly 103. The hand stapler assembly 101 includes at least one staple nail 1013, which has two legs each, a hammer 1011 for striking the staple nail, a block 1015 used to push the staple nails forward, a spring 1017 and a shaft 1019 both used to maintain an appropriate position of the block 1015. The cutter assembly 103 includes a staple plate 1031 for bending the two legs, a knife 1033 for cutting the foreskin of a patient, a member 1038, and a pry device 1035. The member 1038 has a first end 1037 and a second end 1039, and the member 1038 is connected with the knife 1033 at the first end 1037. A user can utilize the stapler apparatus 300 by hand grasping, like the operational usage of a stapler or a pair of scissors. Details are referring to the descriptions of FIGS. 2b-2c.

Now please refer to FIG. 3b, which shows yet another embodiment of the stapler of the present invention. In this embodiment, the stapler apparatus 300 includes a hand stapler assembly 101 and a support assembly 103. A connection device 105 pivotally connects the hand stapler assembly 101 and the support assembly 103. The hand stapler assembly 101 includes at least one staple nail 1013, which has two legs each, a hammer 1011 for striking the staple nail, a block 1015 used to push the staple nails forward, a spring 1017 and a shaft 1019 both used to maintain an appropriate position of the block 1015. The hand stapler assembly 101 also includes a knife 1033 for cutting the foreskin of a patient, a member 1038, and a pry device 1035. The member 1038 has a first end 1037 and a second end 1039, and the member 1038 is connected with the knife 1033 at the first end 1037. The support assembly 103 includes a staple plate 1031 for bending the two legs The difference between this embodiment and the aforementioned embodiment in FIG. 3a is that the knife 1033, the pry device 1035 and the member 1038 are disposed in the hand stapler assembly 101. A user can utilize the stapler apparatus 300 by hand grasping, like the operational usage of a stapler or a pair of scissors, details please referring to the descriptions of FIGS. 2b-2c.

FIGS. 4a, 4b, 4c and 4d are schematic views illustrating the circumcision method using the stapler apparatus of the present invention. Referring to FIG. 4a, a user is prepared to perform a circumcision on a penis 400 of a patient. The stapler apparatus 100 is placed at an operation position (please referring back to the descriptions of FIG. 2a), as the foreskin 20 surrounds the cutter assembly 103. Referring to FIG. 4b, the user utilizes the stapler apparatus 100 to form several cuts 41 on the foreskin 20 and leave several staple nails 1031 near these cuts 41 to stop bleeding (referring to the descriptions of FIGS. 2a-2c). Repeat the stapling and cutting of the foreskin 20 until the user is completely around the penile shaft. Referring now to FIG. 4c, the user can utilizes several forceps 43 to peel up the foreskin 20 to reveal the glans penis 401. Please note that the previously cut foreskin 20 should still be attached to the rest of the outer layer of the skin by the staple nails 1013. Referring now to FIG. 4d, use a pair of scissors 45 to cut around the peeled up foreskin 20 in the same path as the previously made cuts 41. Please make sure not to cut any of the staple nails 1013. Subsequently, the foreskin 20 will be separated into a first part and a second part 402. The circumcision procedure is complete within 5 minutes. It really shortens the operative time and causes much less tissue edema and bleeding during the operation.

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

1. A stapler apparatus for performing surgical operation on a patient with phimosis, the stapler apparatus comprising:
a staple nail having two legs;
a knife for cutting a foreskin of the patient;
a member having a first end and a second end, the member being connected with the knife at the first end; and
a pry device disposed between the first end and the second end,
wherein, when a force is applied to the second end of the member, the knife moves to separate the foreskin of the patient into a first part and a second part, and simultaneously, the force is also applied to the staple nail to make the two legs penetrate the second part and then the two legs are bended to approximate a cut on the second part.

2. The stapler apparatus according to claim 1, further comprising:
a staple plate for bending the two legs; and
a hammer for striking the staple nail.

3. The stapler apparatus according to claim 1, wherein there is no relative movement between the pry device and the staple plate.

4. The stapler apparatus according to claim 1, wherein the knife is a scalpel.

5. The stapler apparatus according to claim 1, wherein the stapler apparatus is made from materials compatible with human bodies.

6. The stapler apparatus according to claim 1, wherein the stapler apparatus further comprises:
a knife mount device for securing the knife at the first end of the member; and
a follow spring used to push the staple forward.

7. The stapler apparatus for performing a circumcision on a patient, the patient having a foreskin, the foreskin being placed at an operation position, the stapler apparatus comprising:
a staple nail, having two legs; a hammer for striking the staple nail;
a staple plate for bending the two legs;
a knife for cutting the portion;
a member having a first end and a second end, the member being connected with the knife at the first end; and
a pry device disposed between the first end and the second end,
wherein, when a force is applied to the second end of the member, the knife moves to separate the foreskin into a first part and a second part, and simultaneously, the force is also applied to the hammer to strike the staple nail to make the two legs penetrate the second part with the staple plate bending the two legs to approximate a cut on the second part to reduce bleeding from the cut.

8. The stapler apparatus according to claim 7, wherein there is no relative movement between the pry device and the staple plate.

9. The stapler apparatus according to claim 7, wherein the knife is a scalpel.

10. The stapler apparatus according to claim 7, wherein the stapler apparatus is made from materials compatible with human bodies.

11. The stapler apparatus according to claim 7, wherein the stapler apparatus further comprises:
a knife mount device for securing the knife at the first end of the member; and
a follow spring to push the staple forward.

12. A method of performing circumcision on patient with a stapler apparatus, the patient having redundant foreskin, the stapler apparatus having a staple nail with two legs, a hammer for striking the staple nail, a staple plate for bending the two legs, a knife for cutting the foreskin, a member and a pry device, the member having a first end and a second end, the member connected with the knife at the first end, the pry device disposed between the first end and the second end, the method comprising:
placing the foreskin at an operation position;
applying a force on the second end of the member, then the knife moving to separate the foreskin into a first part and a second part, and simultaneously, applying the force on the hammer to strike the staple nail to make the two legs penetrate the second part; and
bending the two legs by the staple plate to approximate a cut on the second part to reduce bleeding from the cut.

13. The method according to claim 12, wherein there is no relative movement between the pry device and the staple plate.

14. The method according to claim 12, wherein the knife is a scalpel.

15. The method according to claim 12, wherein the stapler apparatus is made from materials compatible with human bodies.

16. The method according to claim 12, the stapler apparatus further being comprised of the following:
a knife mount device for securing the knife at the first end of the member; and
a follow spring to push the staple forward.
